(19) United States
(12) Patent Application Publication
(10) Pub. No.: US 2022/0175756 A1
(43) Pub. Date: Jun. 9, 2022

(54) APPLICATION OF COMBINATION OF QUINOLINE DERIVATIVE AND IMMUNOMODULATOR IN PREPARATION OF ANTITUMOR DRUGS

(71) Applicants: CHONGQING PHARMACEUTICAL INDUSTRIAL RESEARCH INSTITUTE CO., LTD., Chongqing (CN); YAOPHARMA CO., LTD., Chongqing (CN)

(72) Inventors: Yang ZHANG, Shanghai (CN); Zhengxia CHEN, Shanghai (CN); Jian LI, Shanghai (CN); Shuhui CHEN, Shanghai (CN)

(73) Assignees: CHONGQING PHARMACEUTICAL INDUSTRIAL RESEARCH INSTITUTE CO., LTD., Chongqing (CN); YAOPHARMA CO., LTD., Chongqing (CN)

(21) Appl. No.: 17/437,428

(22) PCT Filed: Mar. 16, 2020
(86) PCT No.: PCT/CN2020/079540

§ 371 Date: Sep. 9, 2021

(30) Foreign Application Priority Data
Mar. 15, 2019 (CN) 201910198729.7

(51) Int. Cl. A61K 31/47; A61K 39/395; A61P 35/00

(52) U.S. Cl. CPC A61K 31/47; A61K 2039/505; A61P 35/00; A61K 39/3955

(57) ABSTRACT

Provided is an application of the combination of a quinoline derivative and an immunomodulator in preparation of antitumor drugs.

This application claims the priority of CN201910198729.7 filed on Mar. 15, 2019.

### FIELD

The present disclosure relates to application of combination of a class of quinoline derivatives with immunomodulators in preparation of antitumor drugs.

### BACKGROUND

Protein tyrosine kinase (PTK) is an enzyme, which phosphorylates tyrosine residues in peptides and proteins, together with ATP being as a substrate. These enzymes are key factors in the regulation of cell signal transduction, such as cell proliferation and differentiation. Specifically, PTK includes receptor tyrosine kinases including hepatocyte growth factors (HGF), platelet derived growth factors (PDGF) and kinases acting in angiogenesis (FGF and VEGF). In addition, it further includes nonreceptor tyrosine kinases including LCK and ABL.

The c-Met protein (also known as hepatocyte growth factor (HGF) receptor) is a transmembrane 190 kDa heterodimer with tyrosine kinase activity, and is encoded by c-Met oncogene. It has been showed that HGF/c-Met signaling pathway is confirmed in various cellular responses, including mitogenic activity, proliferative activity, morphogenetic activity and angiogenic activity. Inhibitors of the HGF/c-Met pathway have significant potential for treating cancers.

ABL is a tyrosine kinase encoded by a proto-oncogene, and the activated ABL is able to promote cell proliferation, differentiation and EMT. In haematomas, it is manly activated through gene fusion such as BCR-ABL. In solid tumors, it is manly activated through gene amplification, overexpression and upstream receptor tyrosine kinases, such as PDGFR and EGFR. TNIK is a serine/threonine kinase, which is capable of bonding to β-catenin/TCF in wnt signaling pathway, activating downstream target genes of wnt signaling and promoting tumor growth. MINK is a member of the STE20 protein kinase family, which is highly expressed in the central nervous system, and capable of activating JNK and p38 signaling pathways.

FGFR is a class of biologically active substances, having functions of transmitting biological signals, regulating cell growth, and participating in tissue repair. In recent years, it has been found that a plurality of FGFR family members take important roles in the occurrence and development of tumors. Fibroblast growth factor receptor (FGFR) is a type of receptor protein capable of specifically binding to fibroblast growth factor (FGF), and FGFRs family includes the following types: FGFR1b, FGFR1c, FGFR2b, FGFR2c, FGFR3b, FGFR3c, and FGFR4. Different subtypes of FGFRs will bind with different FGFs, and the bonding of FGFs and FGFRs causes autophosphorylation of a plurality of intracellular tyrosine residues, and phosphorylated FGFRs activate downstream signaling pathways including MEK/MAPK, PLCy/PKC, PI3K/AKT, STATS, etc. Among them, FGFR4 is highly expressed in liver cancer, colon cancer, gastric cancer, esophageal cancer, and testicular cancer, while FGF19, which specifically binds to FGFR4, is highly expressed in human colon cancer, liver cancer and lung cancer cells, and thus signal abnormalities of specific binding of FGFR4 and FGF19 is an important factor for various tumor occurrence and metastasis.

Vascular endothelial growth factor (VEGF) and platelet derived growth factor (PDGF) play an important role in tumor angiogenesis. They bond with their receptors VEGFR or PDGFR, and transmit signals to the intracellular area, and further generate phosphorylated dimers which activates this signaling pathway, and transmit energy downstream, thereby resulting in uncontrolled tumor cell growth, metastasis, and proliferation.

For targets, such as ABL, C-Met, TNIK, FGFR₁-4, VEGFR (FLT1, KDR, FLT4) shown above, considering from the view point of molecular mechanism in the treatment of tumor cells by PDGFR, targets can produce synergistic complementarity among themselves, reducing escape of tumor cells, reducing drug resistance, and improving therapeutic effects, therefore drugs acting on these targets at the same time are much expected.

### SUMMARY

The present disclosure provides use of a combination of a compound of Formula (I) or a pharmaceutically acceptable salt thereof with an immunomodulator in preparation of a medicament for treating a tumor,

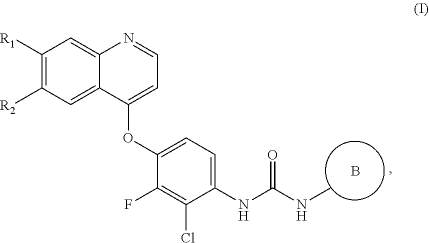

wherein R₁ is selected from C₁₋₆alkoxy optionally substituted by 1, 2 or 3 R;

R₂ is selected from —C(═O)NH₂ and —C(═O)NH—C₁₋₃ alkyl;

ring B is selected from C₃₋₆ cycloalkyl; and

R is selected from F, Cl, Br, I, OH and NH₂.

The present disclosure provides use of a compound of Formula (I) or a pharmaceutically acceptable salt thereof in preparation of a medicament for treating a tumor in combination with an immunomodulator.

The present disclosure provides use of an immunomodulator in preparation of a medicament for treating a tumor in combination with a compound of Formula (I) or a pharmaceutically acceptable salt thereof.

In some embodiments of the present disclosure, the above-mentioned ring B is selected from cyclopropanyl.

In some embodiments of the present disclosure, the above-mentioned R₁ is selected from

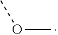

In some embodiments of the present disclosure, the above-mentioned R₂ is selected from —C(═O)NH₂.

In some embodiments of the present disclosure, the above-mentioned compound of Formula (I) or a pharmaceutically acceptable salt thereof is selected from

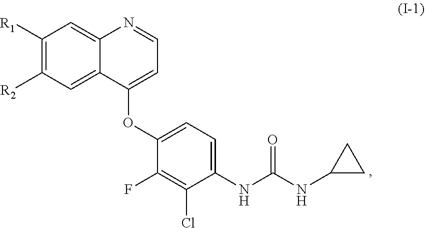

wherein R₁ and R₂ are as defined in the present disclosure.

In some embodiments of the present disclosure, the above-mentioned compound of Formula (I) or pharmaceutically acceptable salt thereof has a structure of the following formula,

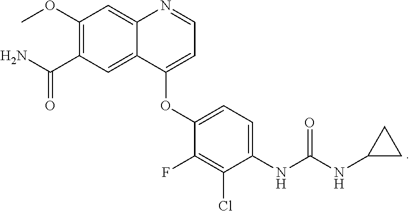 [SMILES: COc1cc2nccc(Oc3ccc(NC(=O)NC4CC4)c(Cl)c3F)c2cc1C(N)=O]

In some embodiments of the present disclosure, the above-mentioned immunomodulator is selected from a PD-1 inhibitor or a PDL-1 inhibitor.

In some embodiments of the present disclosure, the above-mentioned immunomodulator is selected from a PD-1 antibody or a PDL-1 antibody.

In some embodiments of the present disclosure, the above-mentioned immunomodulator is selected from PD-1 antibody.

In some embodiments of the present disclosure, the above-mentioned PD-1 antibody is selected from pembrolizumab, nivolumab, sintilimab, toripalimab, RMP1-14, camrelizumab, tislelizumab and Cemiplimab.

In some embodiments of the present disclosure, the above-mentioned PD-1 antibody is selected from pembrolizumab, nivolumab, sintilimab, toripalimab, RMP1-14, camrelizumab, tislelizumab, spartalizumab, relatlimab+nivolumab (solid tumors), Bristol-Myers Squibb, BCD-100, Biocad, genolimzumab, durvalumab+MEDI-0680 (solid tumor), MedImmune/AstraZeneca pazopanib+pembrolizumab (renal cell cancer), Merck/Novartis, dostarlimab, NANT Hepatocellular Carcinoma Vaccine, BAT-1306, AGEN-2034, JNJ-63723283, GLS-010, MGA-012, AK-104, AK-103, JTX-4014, PF-06801591, HLX-10, RG-7769, PF-06936308, MEDI-5752, PD-1 checkpoint inhibitor (cancer), Sinocelltech, CS-1003, Sym-021, LZM-009, MGD-019, REGN-1979+REGN-2810, BI-754091, XmAb-20717, ABBV-181, PF-06753512, anti-PD1mAb, Fujian Haixi Pharmaceuticals, IBI-318, nivolumab biosimilar, BioXpress Therapeutics, pembrolizumab biosimilar, BioXpress Therapeutics, PD-1 checkpoint inhibitor (cancer), Harvard/Zateras, PD-1 checkpoint inhibitor+A2 aRcheckpoint inhibitor (cancer), Domain Therapeutics/Merck KGaA, PD-1 checkpoint inhibitor (cancer), Waterstone Pharmaceuticals, dual targeting anti-PD-1/LAG-3mAbs (cancer), TESARO, dual targeting anti-PD-1/TIM-3mAbs (cancer), TESARO, STI-1110, ADU-1503 and Cemiplimab.

In some embodiments of the present disclosure, the above-mentioned immunomodulator is selected from PDL-1 antibody.

In some embodiments of the present disclosure, the above-mentioned PDL-1 antibody is selected from Avelumab, atezolizumab and Durvalumab.

In some embodiments of the present disclosure, the above-mentioned PDL-1 antibody is selected from durvalumab, avelumab, atezolizumab, KN-035, durvalumab+tremelimumab (solid tumor), AstraZeneca/MedImmune, CS-1001, bintrafusp alfa, durvalumab+AZD-5069 (SCCHN), AstraZeneca, durvalumab+MEDI-0680 (solid tumor), MedImmune/AstraZeneca, durvalumab+dabrafenib+trametinib (cancer), MedImmune/AstraZeneca, CX-072, BGB-A333, BMS-936559, NANT Colorectal Cancer Vaccine containing aldoxorubicin, NANT Hepatocellular Carcinoma Vaccine, NANT Squamous Cell Carcinoma Vaccine, NANT Triple Negative Breast Cancer Vaccine, NANT Merkel Cell Carcinoma Vaccine, NANT Pancreatic Cancer Vaccine, KL-A167, durvalumab+oleclumab (solid tumor/NSCLC), Medimmune/AstraZeneca, durvalumab+monalizumab (solid tumor/NSCLC), Medimmune/Astrazeneca, SHR-1316, durvalumab+danvatirsen (SCCHN/solid tumor/NSCLC), MedImmune/Astrazeneca, TQB-2450, CK-301, STI-A1014, durvalumab+gefitinib (NSCLC), MedImmune/AstraZeneca, BCD-135, KN-046, INBRX-105, IMC-001, HLX-20, trabectedin+durvalumab (iv, ovarian cancer/soft tissue sarcoma), AstraZeneca/PharmaMar, FAZ-053, durvalumab+AZD-1775 (solid tumor), Medimmune/AstraZeneca, selumetinib+durvalumab (solid tumors), AstraZeneca, FS-118 and LY-3300054.

In some embodiments of the present disclosure, the above-mentioned compound of Formula (I) or pharmaceutically acceptable salt thereof and the immunomodulator are administered separately.

In some embodiments of the present disclosure, the above-mentioned compound of Formula (I) or pharmaceutically acceptable salt thereof and the immunomodulator are administered simultaneously.

In some embodiments of the present disclosure, the compound of Formula (I) or a pharmaceutically acceptable salt thereof is administered, after the above-mentioned immunomodulator.

In some embodiments of the present disclosure, the compound of Formula (I) or a pharmaceutically acceptable salt thereof is administered, before the above-mentioned immunomodulator is administered.

In some embodiments of the present disclosure, the above-mentioned compound of Formula (I) or pharmaceutically acceptable salt thereof is administered before 24 hours (1 day), 2 days, 3 days, 4 days or 5 days of administration of the immunomodulator.

In some embodiments of the present disclosure, compared with single compound of Formula (I) or pharmaceutically acceptable salt thereof, or single immunomodulator, the above-mentioned compound of Formula (I) or pharmaceutically acceptable salt thereof and the above-mentioned immunomodulator treat a tumor in a synergistic way.

The present disclosure further provides a composition comprising: a compound represented by Formula (I) or an isomer or a pharmaceutically acceptable salt thereof, and an immunomodulator.

In some embodiments of the present disclosure, the above-mentioned compound of Formula (I) or pharmaceutically acceptable salt thereof has a structure of the following formula

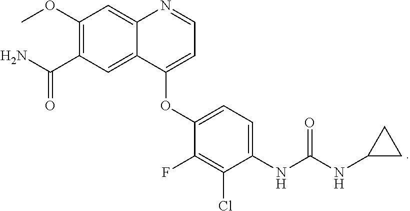 [SMILES: COc1cc2nccc(Oc3ccc(NC(=O)NC4CC4)c(Cl)c3F)c2cc1C(N)=O]

In some embodiments of the present disclosure, the above-mentioned immunomodulator is selected from a PD-1 antibody.

In some embodiments of the present disclosure, use of the above-mentioned composition in preparation of a medicament for treating a tumor disease is provided.

In some embodiments of the present disclosure, the above-mentioned tumor is selected from hepatocellular carcinoma, renal cell carcinoma, endometrial carcinoma, gastric cancer, bile duct cancer, non-small cell lung cancer, melanoma, urinary epithelial cell carcinoma, malignant glioma, esophageal cancer, pancreatic cancer, breast cancer, bowel cancer, lymphadenoma, cervical cancer and brain cancer.

### Definition and Description

Unless otherwise stated, the following terms and phrases used herein are intended to have the following meanings. A specific term or phrase for which no special definition is provided should not be considered uncertain or unclear, but should be understood in its ordinary meaning. When a trade name appears herein, it is meant to refer to the corresponding commodity or the active ingredient thereof. The term “pharmaceutically acceptable” used herein refers to those compounds, materials, compositions and/or dosage forms that within the scope of reliable medical judgment are suitable for contacting with human and animal tissues, without excessive toxicity, irritation, allergic reactions or other problems or complications, and that are commensurate with a reasonable benefit/risk ratio.

The term “pharmaceutically acceptable salt” refers to a salt of the compound of the present disclosure, which is prepared from a compound with specific substituents discovered in the present disclosure and a relatively non-toxic acid or base. When the compound of the present disclosure contains a relatively acidic functional group, a base addition salt can be obtained by contacting the neutral form of the compound with a sufficient amount of base in a pure solution or a suitable inert solvent. Pharmaceutically acceptable base addition salts include sodium salts, potassium salts, calcium salts, ammonium salts, organic ammonium salts or magnesium salts or similar salts. When the compound of the present disclosure contains a relatively basic functional group, an acid addition salt can be obtained by contacting the neutral form of the compound with a sufficient amount of acid in a pure solution or a suitable inert solvent. Examples of pharmaceutically acceptable acid addition salts include inorganic acid salts, said inorganic acids include, for example, hydrochloric acid, hydrobromic acid, nitric acid, carbonic acid, bicarbonate, phosphate, monohydrogen phosphoric acid, dihydrogen phosphate, sulfuric acid, hydrogen sulfate, hydroiodic acid, phosphorous acid, etc.; as well as organic acid salts, wherein the organic acid includes, for example, acetic acid, propionic acid, isobutyric acid, maleic acid, malonic acid, benzoic acid, succinic acid, octanedioic acid, fumaric acid, lactic acid, mandelic acid, phthalic acid, benzenesulfonic acid, p-toluene sulfonic acid, citric acid, tartaric acid and methanesulfonic acid, etc.; and also include salts of amino acids (such as arginine, etc.), and salts of organic acids such as glucuronic acid. Certain specific compounds of the present disclosure contain both basic and acidic functional groups, and thus can be converted into any base or acid addition salt.

The pharmaceutically acceptable salt of the present disclosure can be synthesized from the parent compound containing an acidic or basic functional group by conventional chemical methods. Generally, such salts are prepared by reacting these compounds in free acid or base form with stoichiometric amounts of appropriate bases or acids in water or organic solvents or a mixture of both.

The compounds of the present disclosure may exist in specific geometric or isomeric forms. The present disclosure contemplates all such compounds, including cis and trans isomers, (−)- and (+)-enantiomers, (R)- and (S)-enantiomers, diastereomers, (D)-isomers, (L)-isomers, and the racemic mixture and other mixtures thereof, such as enantiomers or diastereomeric enriched mixtures, which are all within the scope of the present disclosure. Additional asymmetric carbon atoms may be present in a substituent, such as alkyl. All these isomers and their mixtures are included in the scope of the present disclosure.

The optically active (R)- and (S)-isomers and D and L isomers can be prepared by chiral synthesis or chiral reagents or other conventional techniques. In order to obtain an enantiomer of a compound of the present disclosure, it can be prepared by asymmetric synthesis or derivatization with chiral auxiliary agents, in which the resulting mixture of diastereomers is separated, and the auxiliary group is cleaved to provide the pure desired enantiomer. Alternatively, when the molecule contains a basic functional group (such as an amino group) or an acidic functional group (such as a carboxyl group), it can form diastereomeric salts with appropriate optically active acids or bases, and then diastereomers can be resolved by conventional methods known in the art to recover the pure enantiomers. In addition, the separation of enantiomers and diastereomers is usually accomplished through the use of chromatography, which employs a chiral stationary phase and is optionally combined with chemical derivatization (for example, a carbamate is generated from an amine).

The compounds of the present disclosure may contain unnatural proportions of atomic isotopes on one or more of the atoms constituting the compound. For example, compounds can be labeled with radioisotopes, such as tritium (³H), iodine-125 (¹²⁵I) or C-14 (¹⁴C). For another example, deuterated drugs can be formed by replacing hydrogen with deuterium. The bond between deuterium and carbon is stronger than that of ordinary hydrogen and carbon. Compared with non-deuterated drugs, deuterated drugs have reduced toxic side effects and increased drug stability, enhanced efficacy, extended biological half-life and other advantages. All changes in the isotopic composition of the compounds of the present disclosure, whether radioactive or not, are included in the scope of the present disclosure. “Optional” or “optionally” means that the event or condition described later may but not necessarily occur, and the description includes both the situation where the event or condition occurs and the situation where the event or condition does not occur.

The term “substituted” means that any one or more hydrogen atoms on a specific atom are replaced by substituents, including deuterium and hydrogen variants, as long as the valence of the specific atom is normal and the substituted compound is stable. When the substituent is oxygen (i.e. ═O), it means that two hydrogen atoms are replaced. Oxygen substitution does not occur on aromatic groups. The term “optionally substituted” means that the group may be substituted or unsubstituted. Unless otherwise specified, the type and number of substituents can be arbitrary on the basis that they can be chemically realized.

When any variable (such as R) occurs more than once in the composition or structure of a compound, its definition in each case is independent. Thus, for example, if a group is substituted with 0-2 Rs, the group can optionally be substituted with up to two Rs, and in each case R is selected independently. In addition, combinations of substituents and/or variants thereof are only permitted if such combinations result in stable compounds.

When the number of a linking group is 0, such as —(CRR)₀—, it means that the linking group is a single bond.

When one of the variables is selected from a single bond, it means that the two groups connected are directly connected. For example, when L in A-L-Z represents a single bond, it means that the structure is actually A-Z.

When a substituent is vacant, it means that the substituent is absent. For example, when X in A-X is vacant, it means that the structure is actually A. When it is not specified through which atom the listed substituent is connected to the substituted group, such substituents can be bonded at any atom. For example, the pyridyl group as a substituent can be connected to the substituted group through any carbon atom on the pyridine ring.

Unless otherwise specified, when a group has one or more connectable sites, it can connected, via any one or more of its sites, to other groups through chemical bonds. The chemical bond between the sites and other groups may be represented by a straight solid bond ( [symbol] ), a straight dashed bond ( [symbol] ) or a wavy line

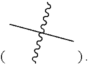

For example, the straight solid bond in —OCH₃ indicates that this group is connected to other groups via the oxygen atom therein; the straight dashed bonds in

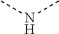

indicate that this group is connected to other groups via the two ends of nitrogen atom therein; and the wavy lines in

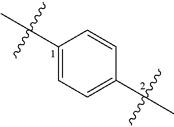 [SMILES: *c1ccc(*)cc1]

indicate that the phenyl group is connected to other groups via the carbon atoms at site 1 and 2 therein.

Unless otherwise specified, the number of atoms in the ring generally was defined as the number of ring members. For example, “5-7 membered ring” refers to a “ring” containing 5-7 atoms arranged in a circle.

Unless otherwise specified, the term “C₁₋₃ alkyl” is used to denote a linear or branched saturated hydrocarbon group composed of 1 to 3 carbon atoms. The C₁₋₃ alkyl group includes C₁₋₂ and C₂₋₃ alkyl groups, etc.; and can be monovalent (such as methyl), divalent (such as methylene) or multivalent (such as methine). Examples of C₁₋₃ alkyl groups include, but are not limited to, methyl (Me), ethyl (Et), propyl (including n-propyl and isopropyl), and the like.

Unless otherwise specified, the term “C₁₋₆ alkoxy” refers to an alkyl group containing 1 to 6 carbon atoms that is attached to the rest of the molecule through an oxygen atom. The C₁₋₆ alkoxy group includes C₁₋₄, C₁₋₃, C₁₋₂, C₂₋₆, C₂₋₄, C₆, C₅, C₄, C₃ alkoxy, etc. Examples of C₁₋₆ alkoxy include but are not limited to methoxy, ethoxy, propoxy (including n-propoxy and isopropoxy), butoxy (including n-butoxy, isobutoxy, s-butoxy and t-butoxy), pentoxy (including n-pentoxy, isopentoxy and neopentoxy), hexoxy and the like.

Unless otherwise specified, “C₃₋₆ cycloalkyl” means a saturated cyclic hydrocarbon group composed of 3 to 6 carbon atoms, which is a monocyclic and bicyclic ring system. The C₃₋₆ cycloalkyl includes C₃₋₅, C₄₋₅ and C₅₋₆ cycloalkyl, etc.; and can be monovalent, divalent or multivalent. Examples of C₃₋₆ cycloalkyl groups include, but are not limited to, cyclopropyl, cyclobutyl, cyclopentyl, cyclohexyl and the like.

Unless otherwise specified, Cₙ₋ₙ₊ₘ or Cₙ-Cₙ₊ₘ includes any specific case of n to n+m carbons, for example, C₁₋₁₂ includes C₁, C₂, C₃, C₄, C₅, C₆, C₇, C₈, C₉, C₁₀, C₁₁, and C₁₂, and also includes any range from n to n+m, for example, C₁₋₁₂ includes C₁₋₃, C₁₋₆, C₁₋₉, C₃₋₆, C₃₋₉, C₃₋₁₂, C₆₋₉, C₆₋₁₂, and C₉₋₁₂, etc. Similarly, n-membered to n+m-membered means that the number of atoms in the ring is from n to n+m, for example, a 3-12-membered ring includes a 3-membered ring, 4-membered ring, 5-membered ring, 6-membered ring, 7-membered ring, 8-membered ring, 9-membered ring, 10-membered ring, 11-membered ring, and 12-membered ring, and also includes any range from n to n+m, for example, 3-12 membered ring includes 3-6 membered ring, 3-9 membered ring, 5-6 membered ring, 5-7 membered ring, 6-7 membered ring, 6-8 membered ring, and 6-10 membered ring and the like.

The term “leaving group” refers to a functional group or atom that can be replaced by another functional group or atom through a substitution reaction (for example, an affinity substitution reaction). For example, representative leaving groups include triflate; chlorine, bromine, iodine; sulfonate, such as mesylate, tosylate, p-bromobenzenesulfonate, p-toluenesulfonate, etc.; acyloxy, such as acetoxy, trifluoroacetoxy, etc.

The term “protecting group” includes, but is not limited to, “amino protecting group”, “hydroxy protecting group” or “thiol protecting group”. The term “amino protecting group” refers to a protecting group suitable for preventing side reactions at the amino nitrogen position. Representative amino protecting groups include, but are not limited to: formyl; acyl, such as alkanoyl (such as acetyl, trichloroacetyl or trifluoroacetyl); alkoxycarbonyl, such as tert-butoxycarbonyl (Boc); arylmethyloxycarbonyl, such as benzyloxycarbonyl (Cbz) and 9-fluorenylmethyloxycarbonyl (Fmoc); arylmethyl, such as benzyl (Bn), trityl (Tr), 1,1-di-(4′-methoxyphenyl)methyl; silyl, such as trimethylsilyl (TMS) and tert-butyldimethylsilyl (TBS) and so on. The term “hydroxyl protecting group” refers to a protecting group suitable for preventing side reactions of hydroxyl group. Representative hydroxy protecting groups include but are not limited to: alkyl groups, such as methyl, ethyl, and tert-butyl; acyl groups, such as alkanoyl (such as acetyl); arylmethyl, such as benzyl (Bn), p-methyloxybenzyl (PMB), 9-fluorenylmethyl (Fm) and diphenylmethyl (diphenylmethyl, DPM); silyl, such as trimethylsilyl (TMS) and tert-butyldimethylsilyl (TBS) and so on.

The compounds of the present disclosure can be prepared by a variety of synthetic methods well known to those skilled in the art, including the specific embodiments listed below, the embodiments formed by their combination with other chemical synthesis methods, and equivalents or alternatives thereof known to those skilled in the art. Preferred embodiments include but are not limited to the examples of the present disclosure.

The solvent used in the present disclosure is commercially available. The present disclosure uses the following abbreviations: aq stands for water; HATU stands for O-(7-azabenzotriazol-1-yl)-N,N,N′,N′-tetramethyluronium hexafluorophosphate; EDC stands for N-(3-dimethylaminopropyl)-N′-ethylcarbodiimide hydrochloride; m-CPBA stands for 3-chloroperoxybenzoic acid; eq stands for equivalent; CDI stands for carbonyl diimidazole; DCM stands for dichloromethane; PE stands for petroleum ether; DIAD stands for diisopropyl azodicarboxylate; DMF stands for N,N-dimethylformamide; DMSO stands for dimethyl sulfoxide; EtOAc stands for ethyl acetate; EtOH stands for ethanol; MeOH stands for methanol; CBz stands for benzyloxycarbonyl, which is an amine protecting group; BOC stands for tert-butoxycarbonyl which is an amine protecting group; HOAc stands for acetic acid; NaCNBH₃ stands for sodium cyanoborohydride; r.t. stands for room temperature; O/N stands for overnight; THF stands for tetrahydrofuran; Boc₂O stands for di-tert-butyl dicarbonate; TFA stands for trifluoroacetic acid; DIPEA stands for diisopropylethylamine; SOCl₂ stands for thionyl chloride; CS₂ stands for carbon disulfide; TsOH stands for p-toluenesulfonic acid; NFSI stands for N-fluoro-N-(benzenesulfonyl)benzenesulfonamide; NCS stands for 1-chloropyrrolidine-2,5-dione; n-Bu₄NF stands for tetrabutylammonium fluoride; iPrOH stands for 2-propanol; mp stands for melting point; LDA stands for lithium diisopropylamide.

Compounds are named according to common rules for naming compounds in the field or by ChemDraw® software, and commercially available compounds are indicated by supplier catalog names.

### Technical Effect

The compounds of the present disclosure exhibited significant anti-tumor activity in combination with PD-1 antibody based in the mouse CT26 tumor model. They significantly enhanced the anti-tumor effect of PD-1 antibody.

### BRIEF DESCRIPTION OF DRAWINGS

FIG. 1 shows the effect on tumor volumes in the mouse CT26 colon tumor model of Example 1.

### DETAILED DESCRIPTION

The following examples are intended to describe the present disclosure in detail, but are not meant to limit the present disclosure in any way. The present disclosure has been described in detail herein, and its specific embodiments are also disclosed. It will be obvious to those skilled in the art that various changes and improvements can be made to the specific embodiments of the present disclosure without departing from the spirit and scope of the present disclosure.

Route A

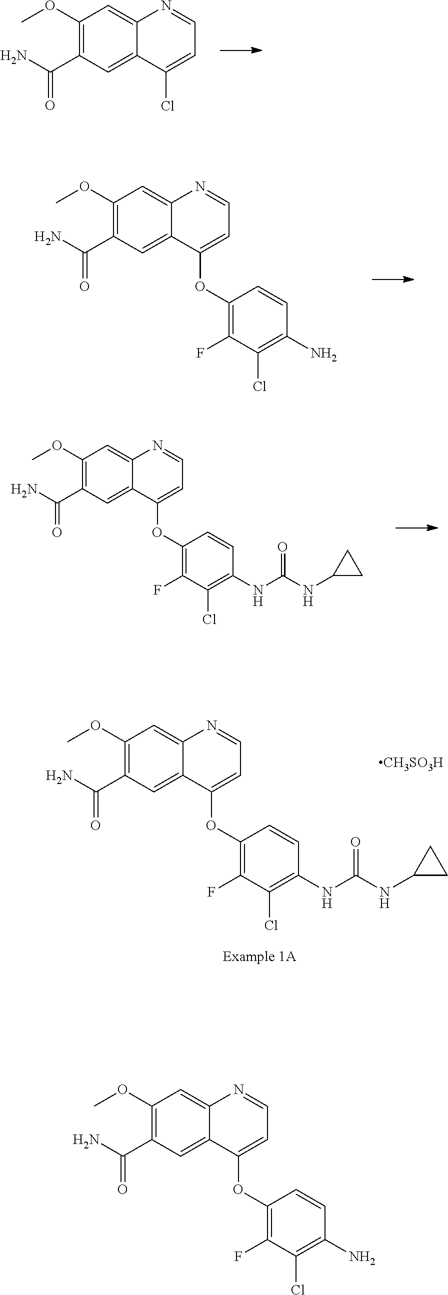 [SMILES: COc1cc2nccc(Cl)c2cc1C(N)=O ; COc1cc2nccc(Oc3ccc(N)c(Cl)c3F)c2cc1C(N)=O ; COc1cc2nccc(Oc3ccc(NC(=O)NC4CC4)c(Cl)c3F)c2cc1C(N)=O ; COc1cc2nccc(Oc3ccc(NC(=O)NC4CC4)c(Cl)c3F)c2cc1C(N)=O ; "·CH3SO3H" ; COc1cc2nccc(Oc3ccc(N)c(Cl)c3F)c2cc1C(N)=O]

At 20-30° C., 4-chloro-7-methoxyquinoline-6-carboxamide (550.0 g) was added into a reaction kettle. At 20-30° C., DMSO (16.5 L) was added into the reaction kettle. At 20-30° C., 2-fluoro-3-chloro-4-aminophenol was added into the reaction kettle. At 20-35° C., sodium tert-butoxide (229 g) was slowly added into the reaction kettle within 10-15 minutes under stirring. The reaction kettle was heated to 96° C. (internal temperature) in 1.5 hours. The reaction was stirred at 96-100° C. for 6.5 hours, until that no 4-amino-3-chloro-2-fluorophenol was left. The reaction was cooled to 20-30° C. Under stirring, 23.1 L water was slowly added into the reaction solution, in the process of which a dark brown solid precipitated out, as the internal temperature was kept below 40° C. The reaction solution was stirred at 30-40° C. for 0.5 hours, cooled to 20-30° C., and filtered. At 20-30° C., the filter cake and 3.5 L water were added into a reaction kettle, stirred at 20-30° C. for 0.5 hours, and filtered. At 20-30° C., the filter cake and 4.0 L water were added into a reaction kettle, stirred at 20-30° C. for 0.5 hour, and filtered. The filter cake was dried in a vacuum dryer at 40° C. for 18 hours (using phosphorus pentoxide as a desiccating agent, and an oil pump to vacuumize). The solid was pulverized to obtain 758 g of off-white solid and dried at 40° C. for 18 hours (using phosphorus pentoxide as a desiccating agent, and an oil pump to vacuumize), and Example 1A was obtained.

LCMS (ESI) m/z: 362.0 [M+1]⁺

¹H NMR (400 MHz, DMSO-d₆) δ ppm 8.68 (br s, 2H), 7.82-7.96 (m, 1H), 7.67-7.82 (m, 1H), 7.46-7.59 (m, 1H), 7.12-7.26 (m, 1H), 6.67-6.80 (m, 1H), 6.43-6.58 (m, 1H), 5.84 (s, 2H), 4.04 (s, 3H).

### Example 1B

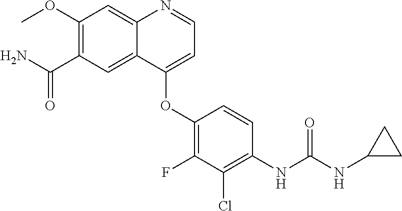 [SMILES: COc1cc2nccc(Oc3ccc(NC(=O)NC4CC4)c(Cl)c3F)c2cc1C(N)=O]

Example 1A (6.05 g) was added into a three-necked flask containing NMP (60 mL). To the reaction system, pyridine (1.32 g), and phenyl chloroformate (5.20 g) were added, and stirred at room temperature (25-30° C.) for 1 hour. After the generation of intermediate was complete, cyclopropylamine (2.84 g) was also added to the reaction system. The reaction solution was stirred at room temperature (25-30° C.) for 0.5 hours. When the reaction was complete, the reaction system was added with 20 mL ethanol, and then with tap water (500 mL) under stirring, resulting in a solid precipitation, and filtered. The filter cake was dried by rotary evaporation under reduced pressure, to obtain a crude product (a soil yellow solid, 5.26 g). The crude product was purified by a chromatography column (DCM:MeOH=20/1-10/1), to obtain a product (a soil yellow solid, 3.12 g). To the product, 4 mL anhydrous ethanol was added at normal temperature and stirred for 18 h, and filtered. The filter cake was washed with 1 mL ethanol, and dried under reduced pressure, to obtain Example 1B. From this compound, the corresponded salt was obtained by adding 1 equivalent of hydrochloric acid or sulfuric acid or methanesulfonic acid in acetone or ethanol solution.

LCMS (ESI) m/z: 445.0 [M+1]⁺

¹H NMR (400 MHz, DMSO-d₆) ppm 8.66-8.71 (m, 2H), 8.12-8.20 (m, 2H), 7.72-7.93 (m, 2H), 7.45 (t, J=9.16 Hz, 1H), 7.28 (d, J=2.76 Hz, 1H), 6.58 (d, J=5.02 Hz, 1H), 4.05 (s, 3H), 2.56-2.64 (m, 1H), 0.38-0.77 (m, 4H).

### Example 1

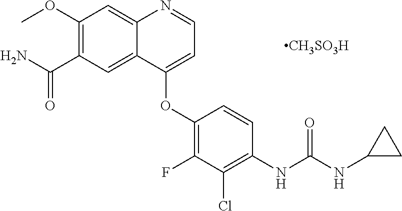 [SMILES: COc1cc2nccc(Oc3ccc(NC(=O)NC4CC4)c(Cl)c3F)c2cc1C(N)=O ; "·CH3SO3H"]

Example 1B (1.5 g, 3.37 mmol) was added to EtOH (45 mL). As the reaction temperature raised to 60° C., CH₃SO₃H (324.07 mg, 3.37 mmol, 240.05 μL) was added dropwise to the reaction solution at this temperature. After the addition was finished, the reaction solution was clear, and allowed to drop to 15-20° C. naturally under stirring at this temperature, and stirred for 2 h at this temperature. A lot of brown solid precipitated out, and was filtered. The filter cake was rinsed with anhydrous ethanol (5 mL). The resulting filter cake was dried at 50° C. by rotary evaporation under reduced pressure, to obtain Example 1 without purification.

LCMS (ESI) m/z: 445.0 [M+1]⁺

¹H NMR (400 MHz, DMSO-d₆) δ ppm 9.02 (d, J=6.53 Hz, 1H), 8.72 (s, 1H), 8.18-8.27 (m, 2H), 7.87-8.03 (m, 2H), 7.65 (s, 1H), 7.53 (t, J=9.03 Hz, 1H), 7.32 (br s, 1H), 7.11 (d, J=6.27 Hz, 1H), 4.08 (s, 3H), 2.55-2.62 (m, 1H), 2.35 (s, 3H), 0.34-0.75 (m, 4H).

### Biological Test Data

### Experimental Example 1: Test on the In Vivo Anti-Tumor Activity of the Compound of the Present Disclosure in an Animal Tumor Model

The experimental purposes was to investigate the in vivo anti-tumor effect of the combination of the test compound with a mouse anti-PD-1 antibody against the mouse colon cancer in CT26 tumor model.

Experimental Method:

Female Balb/c mice were subcutaneously inoculated with CT26 mouse colon carcinoma cell strain, and randomly grouped based on their body weight, after inoculation. And, they were administered as follows.

To the first group (a control group), the administration was started in the afternoon of the day of vaccination in a way that vehicle 1 (10% DMSO+10% Solutol+80% DDH2O) was administered intragastrically twice a day at a dosage of 0.1 mL/10 g body weight each time, and when the tumor had grown to a volume of about 60 mm³ after the inoculation, it started to do administration in a way that vehicle 2 (DPBS) was given twice a week through intraperitoneal injection at a dosage of 0.1 mL/10 g body weight at each time.

To the second group, when the tumor had grown to a volume of about 60 mm³ after the inoculation, it started to do administration in a way that anti-mouse PD-1 antibody (RMP1-14, supplied by BioXcell) was given through intraperitoneal injection twice a week at a dosage of 3 mg/kg body weight at each time.

To the third group: the administration was started in the afternoon of the day of vaccination in a way that Example 1 (co-suspended in 0.5% MC+0.2% Tween 80) was administered intragastrically once a day at a dosage of 10 mg/kg body weight, and when the tumor had grown to a volume of about 60 mm³ after the inoculation, it started to do administration in a way that anti-mouse PD-1 antibody (RMP1-14, supplied by BioXcell) was given through intraperitoneal injection twice a week at a dosage of 3 mg/kg body weight.

During the experiment, the mice were weighed three times a week, and after the tumor formed, the tumor volume was measured three times a week at each time when the body weight was measured, and calculated following the equation: length×width²/2. Tumor proliferation rate was calculated following the equation: Tumor proliferation rate=Tumor volume in the treatment group/Tumor volume in the control group×100%.

The differences between groups were statistically analyzed by Student's t-test, and p<0.05 was considered to be a significant difference.

Experimental Results:

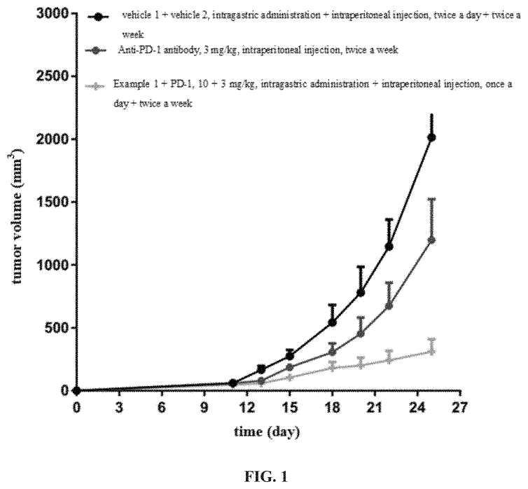

In the mouse CT26 tumor model (as shown in FIG. 1), the combination of Example 1 with PD-1 antibody exhibited significant anti-tumor activity, as its tumor proliferation rate on day 25 was 15.38% (p<0.01), which was significantly higher than that in the single antibody administrated group (the tumor proliferation rate in the single antibody administrated group was 59.44%). The comparison of tumor volumes on day 25 between the combination group and the single antibody administrated group had significant differences (p<0.05), indicating that Example 1 had significantly enhanced anti-tumor effect on PD-1 antibody in this experiment.

1. A method of treating a tumor, comprising administering a combination of a compound of Formula (I) or a pharmaceutically acceptable salt thereof with an immunomodulator to a subject in need thereof, 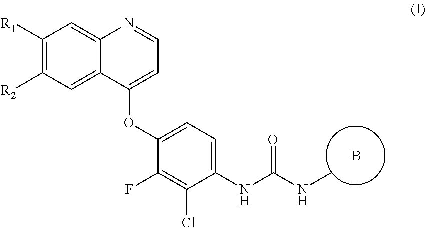
wherein,
R₁ is selected from C₁₋₆ alkoxy optionally substituted by 1, 2 or 3 R;
R₂ is selected from —C(═O)NH₂ and —C(═O)NH—C₁₋₃ alkyl;
ring B is selected from C₃₋₆ cycloalkyl; and
R is selected from F, Cl, Br, I, OH and NH₂.

2. (canceled)

3. (canceled)

4. The method according to claim 1, wherein ring B is selected from cyclopropanyl.

5. The method according to claim 1, wherein R₁ is selected from

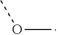

6. The method according to claim 1, wherein R₂ is selected from —C(═O)NH₂.

7. The method according to claim 1, wherein the compound of Formula (I) or a pharmaceutically acceptable salt thereof is selected from

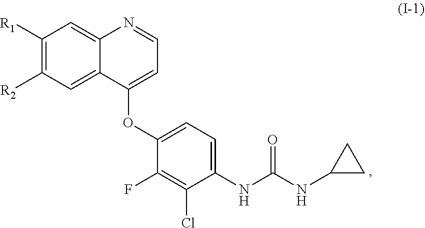

8. The method according to claim 1, wherein the compound of Formula (I) or a pharmaceutically acceptable salt thereof has a structure of the following formula,

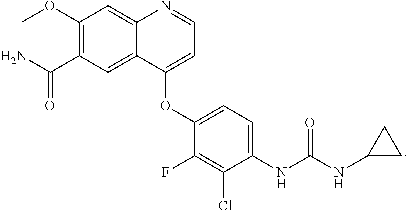 [SMILES: COc1cc2nccc(Oc3ccc(NC(=O)NC4CC4)c(Cl)c3F)c2cc1C(N)=O]

9. The method according to claim 1, wherein the immunomodulator is selected from a PD-1 inhibitor and a PDL-1 inhibitor.

10. The method according to claim 9, wherein the immunomodulator is selected from a PD-1 antibody and a PDL-1 antibody.

11. The method according to claim 10, wherein the immunomodulator is selected from a PD-1 antibody.

12. The method according to claim 11, wherein the PD-1 antibody is selected from pembrolizumab, nivolumab, sintilimab, toripalimab, RMP1-14, camrelizumab, tislelizumab and cemiplimab.

13. The method according to claim 1, wherein the immunomodulator is selected from a PDL-1 antibody.

14. The method according to claim 13, wherein the PDL-1 antibody is selected from avelumab, atezolizumab and durvalumab.

15. The method according to claim 1, wherein the compound of Formula (I) or a pharmaceutically acceptable salt thereof and the immunomodulator are administered separately.

16. The method according to claim 1, wherein the compound of Formula (I) or a pharmaceutically acceptable salt thereof and the immunomodulator are administered simultaneously.

17. The method according to claim 16, wherein the compound of Formula (I) or a pharmaceutically acceptable salt thereof is administered after the immunomodulator is administered.

18. The method according to claim 16, wherein the compound of Formula (I) or a pharmaceutically acceptable salt thereof is administered before the immunomodulator is administered.

19. The method according to claim 18, wherein the compound of Formula (I) or a pharmaceutically acceptable salt thereof is administered before 24 hours (1 day), 2 days, 3 days, 4 days or 5 days of administration of the immunomodulator.

20. The method according to claim 1, wherein the compound of Formula (I) or a pharmaceutically acceptable salt thereof and the immunomodulator treat a tumor in a synergistic way.

21. A composition, comprising:
a compound represented by Formula (I) 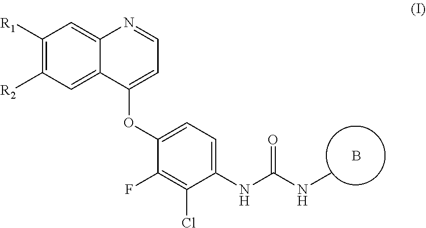
or an isomer or a pharmaceutically acceptable salt thereof, and
an immunomodulator.

22. The composition according to claim 21, wherein the compound of Formula (I) or a pharmaceutically acceptable salt thereof has a structure of the following formula,

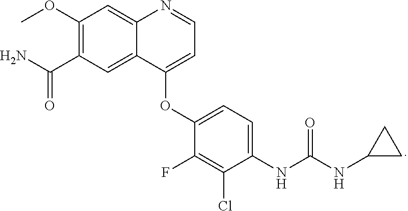 [SMILES: COc1cc2nccc(Oc3ccc(NC(=O)NC4CC4)c(Cl)c3F)c2cc1C(N)=O]

23. The composition according to claim 21, wherein the immunomodulator is selected from a PD-1 antibody.

24. A method of treating a tumor, comprising administering the composition according to claim 21 to a subject in need thereof.

25. The method according to claim 1, wherein the tumor is selected from hepatocellular carcinoma, renal cell carcinoma, endometrial carcinoma, gastric cancer, bile duct cancer, non-small cell lung cancer, melanoma, urinary epithelial cell carcinoma, malignant glioma, esophageal cancer, pancreatic cancer, breast cancer, bowel cancer, lymphadenoma, cervical cancer and brain cancer.
